(19) United States
(12) Patent Application Publication
(10) Pub. No.: US 2005/0170338 A1
(43) Pub. Date: Aug. 4, 2005

(54) Norovirus detection reagent

(75) Inventors: Noriyoshi Masuda, Tokyo (JP); Kiyoshi Yasukawa, Kawasaki-shi (JP); Ryuichi Horie, Zama-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: TOSOH CORPORATION, Shunan-shi (JP)

(21) Appl. No.: 11/049,726

(22) Filed: Feb. 4, 2005

(30) Foreign Application Priority Data
Feb. 4, 2004 (JP) 2004-27802

(51) Int. Cl. C12Q 1/70; C12Q 1/68; C07H 21/04

(52) U.S. Cl. 435/5; 435/6; 536/23.72

(57) ABSTRACT

The present invention provides a combination of oligonucleotides preferable for composing a gene testing reagent capable of detecting all subtypes of norovirus rapidly and with high sensitivity. More specifically, the present invention provides a detection method in which only norovirus is specifically amplified and an oligonucleotide that binds to a specific site of norovirus, by using a primer having a sequence that is homologous or complementary to a base sequence specific for norovirus and is located at a position subject to minimal mutation according to subtype.

### FIELD OF THE INVENTION

Norovirus is known to be a virus that typically causes viral food poisoning. The present invention relates to a norovirus detection reagent used for detecting norovirus in clinical examinations, public health examinations, food evaluations and food poisoning examinations.

### PRIOR ART

Norovirus is a member of the human calicivirus family, and has a genome consisting of a single-strand RNA of about 7000 bases. Norovirus is also referred to as Small Round Structured Virus (SRSV).

Roughly 20% of the cases of food poisoning reported in Japan are estimated to be caused by viruses. Norovirus is detected in about 80% of these cases of viral food poisoning. The main infection source is food, and raw oysters are frequently the problem. In addition, norovirus has also been detected in (sporadic) acute gastroenteritis among infants, and the possibility of person-to-person propagation has been suggested. Since testing for norovirus is therefore an important issue in terms of public health and food quality control, there is a need for the development of a highly-sensitive and rapid testing method capable of detecting all or most of the subtypes, using a gene amplification process. In contrast, detection of norovirus is currently based on observation by electron microscopy. Although this method makes it possible to detect all subtypes, because detection requires an amount of virus of 10⁶ cells/mL or more and the sensitivity of the test is low, specimens are limited to patient stool samples.

### DISCLOSURE OF THE INVENTION

Norovirus is broadly divided into two types consisting of genogroup I (GI) and genogroup II (GII) according to its genotype. Moreover, GI is classified, on the basis of the genotype, into a plurality of subtypes typically represented by Chiba, Desert Shield, Norwalk and Southampton, while GII is classified, on the basis of the genotype, into a plurality of subtypes typically represented by Camberwell, Hawaii, Mexico and Snow Mountain. The base sequence homology among subtypes belonging to GI and among subtypes belonging to GII is about 70%. In addition, the homology between base sequences of GI and GII is about 40-50%.

In order to improve the detectability of a norovirus detection reagent used in a gene amplification process, regarding the regions to be used for primer bindings, there must be at least two regions in a length of at least 20 bases, each region having a base sequence which is common among all subtypes. However, having researched the homology of the following six sequences indicated as norovirus sequences in GenBank (Chiba (No. AB042808), Norwalk (No. NC_{—}001959), Southampton (No. L07418), Camberwell (No. AF145896), Hawaii (No. U07611) and HuCV (No. AY032605)), there are no region of 20 or more bases in which the base sequence is the same among all subtypes.

Under these circumstances, the inventors of the present invention had previously provided norovirus detection methods (Japanese Unexamined Patent Publication No. 2002-51778, Japanese Unexamined Patent Publication No. 2002-153289, Japanese Unexamined Patent Publication No. 2002-218999). However, as the primer base sequences are designed on the basis of the base sequence of each subtype, it is not possible to detect other subtypes. In addition, a norovirus detection reagent using PCR disclosed in Japanese Unexamined Patent Publication No. 2000-300297 can only be used to detect the Norwalk and Snow Mountain subtypes. At present, there is no norovirus detection reagent, used for a gene amplification process, that is capable of detecting all or most of GI or GII subtypes (e.g., capable of detecting at least 80% of the subtypes).

In order to solve this problem, the inventors of the present invention developed a norovirus detection reagent that detects all subtypes of GI and a norovirus detection reagent that detects all subtypes of GII by analyzing the base sequences of the norovirus registered in GenBank as well as the norovirus base sequences determined on their own, and further determining the primer binding regions from these sequences.

Namely, the invention of the present application provides primers used in a norovirus detection reagent and a norovirus detection reagent that uses said primers. In this manner, a first aspect of the present invention for achieving the aforementioned object provides an oligonucleotide useful for detecting genome RNA of norovirus that is an oligonucleotide consisting of at least 10 contiguous bases in any of the sequences shown in SEQ. ID Nos. 1 through 5 and 20 capable of binding to genome RNA of norovirus, a mutant of said oligonucleotide capable of binding to genome RNA of norovirus, e.g. an oligonucleotide in which one or more of the nucleotides in the oligonucleotide consisting of at least 10 contiguous bases in any of the sequences shown in SEQ. ID Nos. 1 through 5 and 20 are deleted, substituted or added, an oligonucleotide that hybridizes under highly stringent conditions with the oligonucleotide consisting of at least 10 contiguous bases in any of the sequences shown in SEQ. ID Nos. 1 through 5 and 20, or a complementary chain of any of said oligonucleotides.

A second aspect of the invention of the present application provides a detection reagent for use in detecting norovirus using an RNA amplification process comprising the steps of:
producing a cDNA with an RNA-dependent DNA polymerase using a specific sequence of norovirus genome RNA as a template, as well as a first primer having a sequence homologous to said specific sequence, and a second primer having a sequence complementary to said specific sequence, wherein either the first primer or second primer has a sequence in which a promoter sequence of an RNA polymerase has been added to its 5′ end, thereby forming a double-strand RNA-DNA;
degrading the RNA portion of said double-strand RNA-DNA by ribonuclease H, thereby producing a single-strand DNA; and
producing a double-strand DNA having said promoter sequence capable of transcribing the RNA composed of the specific sequence of the RNA or the sequence complementary to said specific sequence of the RNA with a DNA-dependent DNA polymerase using said single-strand DNA as a template;
wherein, the double-strand DNA produces an RNA transcription product in the presence of the RNA polymerase, and said RNA transcription product serves as a template for the subsequent cDNA synthesis with the RNA-dependent DNA polymerase;
which reagent comprises,
as the first primer, an oligonucleotide consisting of at least 10 contiguous bases of a sequence in any of the sequences shown in SEQ. ID Nos. 1 through 3, an oligonucleotide in which one or more of the nucleotides in the oligonucleotide consisting of at least 10 contiguous bases in any of the sequences shown in SEQ. ID Nos. 1 through 3 are deleted, substituted or added and is capable of specifically binding to a sequence complementary to the specific sequence, or an oligonucleotide capable of hybridizing under highly stringent conditions with an oligonucleotide consisting of at least 10 contiguous bases in any of the sequences shown in SEQ. ID Nos. 1 through 3 and is capable of specifically binding to a sequence complementary to the specific sequence; and
as the second primer, an oligonucleotide consisting of at least 10 contiguous bases of the sequence in any of the sequences shown in SEQ. ID No. 4 or 20, an oligonucleotide in which one or more of the nucleotides in the oligonucleotide consisting of at least 10 contiguous bases in any of the sequences shown in SEQ. ID No. 4 or 20 are deleted, substituted or added and is capable of specifically binding to the sequence complementary to said specific sequence, or an oligonucleotide capable of hybridizing under highly stringent conditions with an oligonucleotide consisting of at least 10 contiguous bases in any of the sequences shown in SEQ. ID No. 4 or 20 and is capable of specifically binding to a sequence complementary to the specific sequence.

Highly stringent conditions refer to hybridization conditions, for example, like those indicated in the following examples consisting of carrying out a hybridization at a temperature of 43° C. in the presence of 60 mM Tris, 17 mM magnesium chloride, 120 mM potassium chloride and 1 mM DTT.

Furthermore, in the case of intending to detect an RNA complementary to the RNA derived from norovirus, an oligonucleotide having a sequence complementary to the aforementioned first primer with its sequence from the 5′ end to the 3′ end being reversed should be used as the first primer, an oligonucleotide having a sequence complementary to the aforementioned second primer with its sequence from the 5′ end to the 3′ end being reversed should be used as the second primer.

Preferably, the aforementioned RNA amplification process is carried out in the presence of a cleaving oligonucleotide that cleaves the aforementioned target RNA at the 5′ end of the aforementioned specific sequence and has a sequence complementary to the region adjacent to and overlapping with the 5′ end of said specific sequence.

Preferably, the aforementioned first primer is an oligonucleotide consisting of a sequence shown in SEQ. ID Nos. 1 through 3.

Moreover, the aforementioned second primer is preferably an oligonucleotide consisting of a sequence shown in SEQ. ID No. 4 or 20.

In another preferable embodiment, the aforementioned RNA amplification process is carried out in the presence of an oligonucleotide labeled with an intercalator fluorescent pigment, and the detection of norovirus is carried out by measuring the fluorescent intensity of the reaction solution. Here, the sequence of said oligonucleotide is complementary to at least a portion of the sequence of the aforementioned RNA transcription product and, in the situation where complementary binding of said oligonucleotide to said RNA transcription product occurs, the fluorescent properties of the reaction solution change in comparison with the situation where no complex is formed.

Preferably, the aforementioned oligonucleotide labeled with an intercalator fluorescent pigment consists of at least 10 contiguous bases in the sequence shown in SEQ. ID No. 5. The following provides a detailed explanation of the present invention.

### BEST MODE FOR CARRYING OUT THE INVENTION

In the present invention, an oligonucleotide consisting of at least 10 contiguous bases in any of the sequences shown in SEQ. ID Nos. 1 through 5 and 20 or a mutant thereof can be used as a primer. Here, SEQ. ID Nos. 1 through 3, 4 and 20 are regions that are close to each other. Thus, in the case of carrying out PCR, for example, examples of combinations include a combination of any of SEQ. ID Nos. 1 through 3 and SEQ. ID No. 4 or 20, a combination of any of SEQ. ID No. 1 through 3 and SEQ. ID No. 5, or a combination of a complementary chain of SEQ. ID No. 5 and SEQ. ID No. 4 or 20. This applies similarly to other DNA amplification methods other than PCR.

Another embodiment of the present invention includes, for example, the NASBA method, 3SR method or TRC method (see, e.g., Japanese Unexamined Patent Publication No. 2000-014400), which amplifies an RNA sequence of norovirus by the concerted action of reverse transcriptase and RNA polymerase (by reacting them under conditions by which the reverse transcriptase and RNA polymerase act in concert). Here, although there are no particular limitations on temperature, it is preferably 35 to 50° C.

In the aforementioned embodiment of the invention of the present application, it is necessary for a target RNA to be cleaved at the 5′ end of a specific sequence. A preferable method for cleaving the target RNA in this manner preferably consists of cleaving the target RNA with ribonuclease H or the like by adding an oligonucleotide having a sequence complementary to the region adjacent to and overlapping with the 5′ end of the specific sequence (cleaving oligonucleotide). In said cleaving oligonucleotide, the 3′ end hydroxyl group is preferably chemically modified, for example, aminated, in order to suppress an elongation reaction from the 3′ end.

Although the amplification product obtained in the aforementioned nucleic acid amplification method can be detected with a known nucleic acid detection method, in a preferable embodiment, the aforementioned nucleic acid amplification is preferably carried out in the presence of an oligonucleotide labeled with an intercalator fluorescent pigment followed by measurement of the change in the fluorescent properties of the reaction solution. In said oligonucleotide, as the intercalator fluorescent pigment is bound to the phosphorous atom in the oligonucleotide by means of a linker, the intercalator portion that forms a double strand with the target nucleic acid (complementary nucleic acid) intercalates to the double strand portion resulting in a change in fluorescent properties, thereby resulting in the characteristic of not requiring separation and analysis (Ishiguro, T. et al. (1996) Nucleic Acid Res. 24 (24) 4992-4997).

The sequence bound by said oligonucleotide may be any sequence that is amplified in the genome RNA of norovirus, and although there are no particular limitations thereon, a sequence consisting of at least 10 contiguous bases in the sequence shown in SEQ. ID No. 5 is preferable. In addition, the hydroxyl group at the 3′ end of said oligonucleotide is preferably chemically modified (such as by an addition of glycolic acid) to suppress the elongation reaction which may occur by using said oligonucleotide as a primer.

As a result, norovirus RNA can be amplified and detected in a single tube, at a constant temperature and in a single step, rapidly and with high sensitivity, thereby facilitating application to automation.

The detection method of the invention of the present application is useful for detecting all GI subtypes or all GII subtypes of norovirus both rapidly and with high sensitivity.

Although the following provides a more detailed explanation of the invention of the present application through examples, the present invention is not limited by these examples.

### EXAMPLE 1

In order to show that combination (a) shown in Table 1 is capable of detecting all subtypes of GI and that combinations (b) and (c) are capable of detecting all subtypes of GII, DNA (hereafter, referred to as “artificial standard DNA)” and RNA (hereafter, referred to as “artificial standard RNA”) respectively corresponding to four subtypes of GI and four subtypes of GII were prepared using the methods shown in (1) to (8) below. The artificial standard RNA was measured using the methods shown in (9) to (12).

(1) Artificial standard DNA was designed so as to contain an oligonucleotide binding region used in combinations (a) through (c) based on the base sequence of the Chiba subtype of norovirus (GenBank No. AB042808), and then artificial standard RNA consisting of 90 bases (NV1ART-C, SEQ. ID No. 6) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(2) Artificial standard DNA was designed so as to contain an oligonucleotide binding region used in combinations (a) through (c) based on the base sequence of the Desert Shield subtype of norovirus (GenBank No. U04469), and then artificial standard RNA consisting of 90 bases (NV1ART-D, SEQ. ID No. 7) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(3) Artificial standard DNA was designed so as to contain an oligonucleotide binding region used in combinations (a) through (c) based on the base sequence of the Norwalk subtype of norovirus (GenBank No. NC 001959), and then artificial standard RNA consisting of 90 bases (NV1ART-N, SEQ. ID No. 8) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(4) Artificial standard DNA was designed so as to contain an oligonucleotide binding region used in combinations (a) through (c) based on the base sequence of the Southampton subtype of norovirus (GenBank No. L07418), and then artificial standard RNA consisting of 90 bases (NV1ART-S, SEQ. ID No. 9) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(5) Artificial standard DNA was designed so as to contain an oligonucleotide binding region used in combinations (a) through (c) based on the base sequence of the Camberwell subtype of norovirus (GenBank No. AF145896), and then artificial standard RNA consisting of 90 bases (NV2ART-C, SEQ. ID No. 10) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(6) Artificial standard DNA was designed so as to contain an oligonucleotide binding region used in combinations (a) through (c) based on the base sequence of the Hawaii subtype of norovirus (GenBank No. U07611), and then artificial standard RNA consisting of 90 bases (NV2ART-H, SEQ. ID No. 11) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(7) Artificial standard DNA was designed so as to contain an oligonucleotide binding region used in combinations (a) through (c) based on the base sequence of the Mexico subtype of norovirus (GenBank No. U22498), and then artificial standard RNA consisting of 90 bases (NV2ART-M, SEQ. ID No. 12) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(8) Artificial standard DNA was designed so as to contain an oligonucleotide binding region used in combinations (a) through (c) based on the base sequence of the Snow Mountain subtype of norovirus, and then artificial standard RNA consisting of 90 bases (NV2ART-S, SEQ. ID No. 13) was prepared by in vitro transcription. This standard RNA was used as the sample. After quantifying it by UV absorption at 260 nm, the RNA sample was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(9) 20 μL of a reaction solution having the composition indicated below were dispensed into 0.5 mL PCR tubes (Individual Dome Cap PCR Tubes, SSI) followed by the addition of 5 μL of the RNA samples prepared in (1) through (8) thereto. Furthermore, solutions were prepared so that the combinations of the first primer, the second primer and the cleaving oligonucleotide were combined as shown in Table 1.

Composition of Reaction Solution (concentrations are shown as the concentration in the final reaction solution volume of 30 μL)
60 mM Tris-HCl buffer (pH 8.6)
17 mM magnesium chloride
120 mM potassium chloride
6 U RNase inhibitor
1 mM DTT
0.25 mM each of dATP, dCTP, dGTP and dTTP
3.6 mM ITP
3.0 mM each of ATP, CTP, GTP and UTP
0.16 μM cleaving oligonucleotide
1.0 μM first primer
1.0 μM second primer
25 nM oligonucleotide labeled with intercalator pigment (YO—NV—S-G, SEQ ID. No. 5; labeled with the intercalator fluorescent pigment between the 12th “C” and 13th “A” from the 5′ end, and the hydroxyl group on its 3′ end being modified with a glycol group.)
13% DMSO
Distilled water for adjusting volume

(10) After incubating the aforementioned reaction solution at 43° C. for 2 minutes, 5 μL of an enzyme solution having the composition indicated below and pre-heated for 2 minutes at 43° C. were added.

Composition of Enzyme Solution (values shown indicate the values for a final reaction solution volume of 30 μL)
2.0% sorbitol
3.6 μg bovine serum albumin
142 U T7 RNA polymerase (Invitrogen)
6.4 U AMV reverse transcriptase (LifeScience)
Distilled water for adjusting volume

(11) Subsequently, the reaction solution in each of the PCR tubes was measured, over time, at an excitation wavelength of 470 nm and fluorescent wavelength of 520 nm, while being incubated at 43° C., using a fluorescent spectrophotometer equipped with a temperature control function and capable of directly measuring the PCR tubes.

(12) The results for the rise time (the time required for the ratio in the fluorescence increase to reach 1.2 times the sum of the negative control sample's average value plus 3 standard deviations) of the norovirus artificial standard RNA when using each oligonucleotide combination are shown in Table 2. Four standard RNA samples of GI were shown to be detected within 30 minutes with combination (a), while four standard RNA samples of GII were shown to be detected within 30 minutes with combinations (b) and (c).

TABLE 1

| Combination | 1st oligo. | 1st oligo. | 3rd oligo. |
|---|---|---|---|
| (a) | NV1-3SM − 9 | NV1-3FM − 9 | NV2-7RM |
| (b) | NV2-3SM | NV2-3FM | NV2-7RM |
| (c) | NV2-3S20 + 3 | NV2-3F20 + 3 | NV2-7RM |

Table 1 shows the combinations of the first primer, the second primer and the cleaving oligonucleotide used in the experimental system. The hydroxyl group on the 3′ end of the cleaving oligonucleotide base sequence was aminated in the combinations of oligonucleotides for detecting norovirus.

The region from the 1st “A” to the 22nd “A” from the 5′ end of the base sequence of the first primer is a T7 promoter region, and the subsequent region from the 23rd “G” to the 28th “A” is an enhancer sequence. Furthermore, combination (a) is designed using mainly GI norovirus as the target, while combinations (b) and (c) are designed using mainly GII norovirus as the target.

| | |
|---|---|
| Cleaving oligonucleotides: | |
| NV1-3SM-9 | (SEQ. ID No. 14) |
| NV2-3SM | (SEQ. ID No. 15) |
| NV2-3S20 + 3 | (SEQ. ID No. 16) |
| First primer: | |
| NV1-3FM-9 | (SEQ. ID No. 17) |
| NV2-3FM | (SEQ. ID No. 18) |
| NV2-3F20 + 3 | (SEQ. ID No. 19) |
| Second primer: | |
| NV2-7RM | (SEQ. ID No. 4) |

TABLE 2

| | | Rise time (min) | | |
|---|---|---|---|---|
| Artificial RNA | GeneGroup | Combination | Combination | Combination |
| Abbreviation | (GI or GII) | (a) | (b) | (c) |
| NV1ART-C | GI | 19.9 | | |
| NV1ART-D | GI | 22.9 | | |
| NV1ART-N | GI | 18.9 | | |
| NV1ART-S | GI | 16.6 | | |
| NV2ART-C | GII | | 15.5 | 19.3 |
| NV2ART-H | GII | | 15.7 | 19.2 |
| NV2ART-M | GII | | 21.2 | 19.4 |
| NV2ART-S | GII | | 16.3 | 18.2 |

Table 2 shows the results of measuring norovirus artificial standard RNA (four samples each for GI and GII) using the combinations of oligonucleotides shown in Table 1. Among the combinations of oligonucleotides shown in Table 1, combination (a) detected the four standard RNA samples of GI within 30 minutes, while combinations (b) and (c) detected the four standard RNA samples of GII within 30 minutes.

### EXAMPLE 2

Artificial standard RNA of four GI subtypes were measured according to the methods shown in (1) through (8) below in order to show that one of the combinations of oligonucleotides of the invention of the present application is capable of detecting all subtypes of GI.

(1) The Chiba subtype of artificial standard RNA (NV1ART-C, SEQ. ID No. 6) was used as the sample in the same manner as Example 1. After quantifying it by UV absorption at 260 nm, the RNA was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)). (2) The Desert Shield subtype of artificial standard RNA (NV1ART-D, SEQ. ID No. 7) was used as the sample in the same manner as Example 1. After quantifying it by UV absorption at 260 nm, the RNA was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(3) The Norwalk subtype of artificial standard RNA (NV1ART-N, SEQ. ID No. 8) was used as the sample in the same manner as Example 1. After quantifying it by UV absorption at 260 nm, the RNA was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(4) The Southampton subtype of artificial standard RNA (NV1ART-S, SEQ. ID No. 9) was used as the sample in the same manner as Example 1. After quantifying it by UV absorption at 260 nm, the RNA was diluted to 10⁶ copies/5 μL with an RNA diluent (10 mM Tris-HCl (pH 8.0), 1 mM EDTA, 5 mM DTT, 0.25 U/μL RNase inhibitor (Takara Bio)).

(5) 20 μL of a reaction solution having the composition indicated below were dispensed into 0.5 mL PCR tubes (Individual Dome Cap PCR Tubes, SSI) followed by the addition of 5 μL of the RNA samples prepared in (1) through (4) thereto.

Composition of Reaction Solution (concentrations are shown as the concentration in the final reaction solution volume of 30 μL)
60 mM Tris-HCl buffer (pH 8.6)
17.2 mM magnesium chloride
120 mM potassium chloride
6 U RNase inhibitor
1 mM DTT
0.25 mM each of DATP, dCTP, dGTP and dTTP
3.6 mM ITP
3.0 mM each of ATP, CTP, GTP and UTP
0.16 μM cleaving oligonucleotide (NV1-3SM-9, SEQ. ID No. 14, hydroxyl group on 3′ end being aminated)
1.0 μM first primer (NV1-3FM-9, SEQ. ID No. 17)
1.0 μM second primer (NV1-7RM18-1, SEQ. ID No. 20)
15 nM oligonucleotide labeled with intercalator pigment (YO—NV—S-G, SEQ ID. No. 5; labeled with the intercalator fluorescent pigment between the 12th “C” and 13th “A” from the 5′ end, and the hydroxyl group on its 3′ end being modified with a glycol group.)
13% DMSO
Distilled water for adjusting volume

(6) After incubating the aforementioned reaction solution at 43° C. for 2 minutes, 5 μL of an enzyme solution having the composition indicated below and pre-heated for 2 minutes at 43° C. were added.

Composition of Enzyme Solution (values shown indicate the values for a final reaction solution volume of 30 μL)
2.0% sorbitol
3.6 μg bovine serum albumin
142 U T7 RNA polymerase (Invitrogen)
6.4 U AMV reverse transcriptase (LifeScience)
Distilled water for adjusting volume

(7) Subsequently, the reaction solution in each of the PCR tubes was measured, over time, at an excitation wavelength of 470 nm and fluorescent wavelength of 520 nm, while being incubated at 43° C., using a fluorescent spectrophotometer equipped with a temperature control function and capable of directly measuring the PCR tubes.

(8) The results for the rise time (the time required for the ratio in the fluorescence increase to reach 1.2 times the sum of the negative control sample's average value plus 3 standard deviations) of the norovirus artificial standard RNA are shown in Table 3. Four standard RNA samples of GI were shown to be detected within 30 minutes by using a combination of oligonucleotides of the invention of the present application.

TABLE 3

| Artificial RNA | GeneGroup | Rise Time |
|---|---|---|
| Abbreviation | (GI or GII) | [min.] |
| NV1ART-C | GI | 12.3 |
| NV1ART-D | GI | 24.2 |
| NV1ART-N | GI | 13.1 |
| NV1ART-S | GI | 11.4 |

Table 3 shows the results of measuring norovirus artificial standard RNA (four samples for GI) using the combinations of oligonucleotides used in Example 2. The combinations of oligonucleotides used in Example 2 detected the four standard RNA samples of GI within 30 minutes.

### INDUSTRIAL APPLICABILITY

As has been explained above, the detection method of the invention of the present application is effective for detecting all GI subtypes and all GII subtypes of norovirus rapidly and with high sensitivity. The oligonucleotide of the invention of the present application is not limited to that of the base sequences shown in the sequence listings (having 18 to 23 bases), but rather can be a nucleotide comprised of at least 10 contiguous bases in those sequences. This is because it is clear that a base sequence of about 10 bases is sufficient for ensuring specificity to a target nucleic acid of a primer or probe under comparatively low-temperature (preferably 43° C.) conditions.

It will be appreciated by those skilled in the art that while the invention has been described above in connection with particular embodiments and examples, the invention is not necessarily so limited and that numerous other embodiments, examples, uses, modifications and departures from the embodiments, examples and use may be made without departing from the inventive scope of this application.

1. An oligonucleotide useful for detecting genome RNA of norovirus and is capable of binding to genome RNA of norovirus, wherein the oligonucleotide consists of at least 10 contiguous bases in any of the sequences shown in SEQ. ID Nos. 1 through 5 and 20, a sequence in which one or more of the nucleotides in SEQ. ID Nos. 1 through 5 and 20 are deleted, substituted or added, or a sequence which is the complementary chain of any of the sequences shown in SEQ. ID Nos. 1 through 5 and 20.

2. A detection reagent for use in detecting norovirus using an RNA amplification process comprising the steps of:
producing a cDNA with an RNA-dependent DNA polymerase using a specific sequence of norovirus genome RNA as a template, as well as a first primer having a sequence homologous to said specific sequence, and a second primer having a sequence complementary to said specific sequence, thereby forming a double-strand RNA-DNA, wherein either the first primer or the second primer has a sequence in which a promoter sequence of an RNA polymerase has been added to its 5′ end;
degrading the RNA portion of said double-strand RNA-DNA by ribonuclease H, thereby producing a single-strand DNA; and
producing a double-strand DNA having said promoter sequence capable of transcribing the RNA composed of the specific sequence of the RNA or the sequence complementary to said specific sequence of the RNA with a DNA-dependent DNA polymerase using said single-strand DNA as a template;
wherein, the double-strand DNA, produces an RNA transcription product in the presence of the RNA polymerase, and said RNA transcription product serves as a template for the subsequent cDNA synthesis with the RNA-dependent DNA polymerase;
which reagent comprises,
as the first primer, an oligonucleotide consisting of at least 10 contiguous bases in any of the sequences shown in SEQ. ID Nos. 1 through 3, or an oligonucleotide in which one or more of the nucleotides in the oligonucleotide consisting of at least 10 contiguous bases in any of the sequences shown in SEQ. ID Nos. 1 through 3 are deleted, substituted or added and is capable of specifically binding to the sequence complementary to said specific sequence; and
as the second primer, an oligonucleotide consisting of at least 10 contiguous bases in any of the sequences shown in SEQ. ID No. 4 or 20, or an oligonucleotide in which one or more of the nucleotides in the oligonucleotide consisting of at least 10 contiguous bases in any of the sequences shown in SEQ. ID No. 4 or 20 are deleted, substituted or added and is capable of specifically binding to said specific sequence.

3. The detection reagent according to claim 2 wherein the first primer is an oligonucleotide consisting of any of the sequences shown in SEQ. ID Nos. 1 through 3.

4. The detection reagent according to claim 2 wherein the second primer is an oligonucleotide consisting of a sequence shown in SEQ. ID No. 4 or 20.

5. The detection reagent according to claim 2, wherein the RNA amplification process is carried out in the presence of an oligonucleotide that has been labeled with an intercalator fluorescent pigment, and the detection of norovirus is carried out by measuring the fluorescent intensity of the reaction solution; wherein the sequence of said oligonucleotide is complementary to at least a portion of the sequence of the RNA transcription product, and in the situation where complementary binding of said oligonucleotide to said RNA transcription product occurs, the fluorescent properties of the reaction solution change in comparison with the situation where no complex is formed.

6. The detection reagent according to claim 5 wherein the oligonucleotide labeled with the intercalator fluorescent pigment consists of at least 10 contiguous bases of the sequence shown in SEQ. ID No. 5.

7. The detection reagent according to claim 3, wherein the RNA amplification process is carried out in the presence of an oligonucleotide that has been labeled with an intercalator fluorescent pigment, and the detection of norovirus is carried out by measuring the fluorescent intensity of the reaction solution; wherein the sequence of said oligonucleotide is complementary to at least a portion of the sequence of the RNA transcription product, and in the situation where complementary binding of said oligonucleotide to said RNA transcription product occurs, the fluorescent properties of the reaction solution change in comparison with the situation where no complex is formed.

8. The detection reagent according to claim 7 wherein the oligonucleotide labeled with the intercalator fluorescent pigment consists of at least 10 contiguous bases of the sequence shown in SEQ. ID No. 5.

9. The detection reagent according to claim 4, wherein the RNA amplification process is carried out in the presence of an oligonucleotide that has been labeled with an intercalator fluorescent pigment, and the detection of norovirus is carried out by measuring the fluorescent intensity of the reaction solution; wherein the sequence of said oligonucleotide is complementary to at least a portion of the sequence of the RNA transcription product, and in the situation where complementary binding of said oligonucleotide to said RNA transcription product occurs, the fluorescent properties of the reaction solution change in comparison with the situation where no complex is formed.

10. The detection reagent according to claim 9 wherein the oligonucleotide labeled with the intercalator fluorescent pigment consists of at least 10 contiguous bases of the sequence shown in SEQ. ID No. 5.


SEQUENCE LISTING (20 sequences)

SEQ ID NO: 1 - DNA, 23 bp, Artificial Sequence
  Synthetic DNA
ttgtggacaggagatcgctatct

SEQ ID NO: 2 - DNA, 23 bp, Artificial Sequence
  Synthetic DNA
acgtgggagggcgatcgcaatct

SEQ ID NO: 3 - DNA, 20 bp, Artificial Sequence
  Synthetic DNA
tgggagggcgatcgcaatct

SEQ ID NO: 4 - DNA, 18 bp, Artificial Sequence
  Synthetic DNA
tctaatccaggggtctat

SEQ ID NO: 5 - DNA, 18 bp, Artificial Sequence
  Synthetic DNA
gacgccatcttcatttac

SEQ ID NO: 6 - RNA, 90 nt, Artificial Sequence
  Synthetic RNA
gaauacaacgcuuccaugaucucggauuguggacaggagaucgcaaucuccguaaaugaugauggcgucugauugaucccuggauaauca

SEQ ID NO: 7 - RNA, 90 nt, Artificial Sequence
  Synthetic RNA
gaauacaacgcuuccaugaucugagcauguggacaggggaucgcgaucucuguaaaugaugauggcgucugauugaccccuggauaauga

SEQ ID NO: 8 - RNA, 90 nt, Artificial Sequence
  Synthetic RNA
gaauacaacgcuuccaugaccucggauuguggacaggagaucgcgaucuucguaaaugaugauggcgucuuauugaucccuggauaauua

SEQ ID NO: 9 - RNA, 90 nt, Artificial Sequence
  Synthetic RNA
gaauacaacgguuccaugaccuugguuuguggacaggagaucgcaaucucuguaaaugaugauggcgucugauugaucccuggauuguua

SEQ ID NO: 10 - RNA, 89 nt, Artificial Sequence
  Synthetic RNA
gaauacaaagauucucagaucugagcacgugggagggcgaucgcaaucugugugaaugaagauggcgucgaauugaccccuggauugaa

SEQ ID NO: 11 - RNA, 90 nt, Artificial Sequence
  Synthetic RNA
gaauacaaagauucucagaccugagcacgugggagggcgaucgcaaucugugugaaugaagauggcgucggauagaccccuggauuagaa

SEQ ID NO: 12 - RNA, 90 nt, Artificial Sequence
  Synthetic RNA
gaauacaaagguucucagaucuaagcacaugggagggcgaucgcaaucugugugaaugaagauggcgucgaauugaucccuggauuauga

SEQ ID NO: 13 - RNA, 90 nt, Artificial Sequence
  Synthetic RNA
gaauacaaagauucucggauuugagcacgugggagggcgaucgcaaucuuugugaaugaagauggcgucguauagacccuuggauuagag

SEQ ID NO: 14 - DNA, 24 bp, Artificial Sequence
  Synthetic DNA
ccacaatccgagatcatggaagcg

SEQ ID NO: 15 - DNA, 24 bp, Artificial Sequence
  Synthetic DNA
ccacgtgctcagatctgagaatct

SEQ ID NO: 16 - DNA, 20 bp, Artificial Sequence
  Synthetic DNA
ctcccatgtgcttaagtccg

SEQ ID NO: 17 - DNA, 51 bp, Artificial Sequence
  Synthetic DNA
aattctaatacgactcactatagggagattgtggacaggagatcgctatct

SEQ ID NO: 18 - DNA, 51 bp, Artificial Sequence
  Synthetic DNA
aattctaatacgactcactatagggagaacgtgggagggcgatcgcaatct

SEQ ID NO: 19 - DNA, 48 bp, Artificial Sequence
  Synthetic DNA
aattctaatacgactcactatagggagatgggagggcgatcgcaatct

SEQ ID NO: 20 - DNA, 18 bp, Artificial Sequence
  Synthetic DNA
gattatccagggatcaat